(19) United States
(12) Patent Application Publication
(10) Pub. No.: US 2008/0193588 A1
(43) Pub. Date: Aug. 14, 2008

(54) METHOD OF TREATING FOOD WASTE USING ACIDOGENIC KOJI MOLD

(75) Inventor: Masahiro Yamamoto, Kagoshima (JP)

Correspondence Address: PEARNE & GORDON LLP, 1801 EAST 9TH STREET, SUITE 1200, CLEVELAND, OH, 44114-3108, US

(73) Assignees: Masahiro Yamamoto, Kagoshima (JP); Biogenkoji Research Institute, Kagoshima (JP)

(21) Appl. No.: 12/019,052

(22) Filed: Jan. 24, 2008

(30) Foreign Application Priority Data
Jan. 24, 2007 (JP) 2007-13408

(51) Int. Cl. A23K 1/00; A23L 3/3571; A23L 1/28; C12N 1/14; A23L 1/0522

(52) U.S. Cl. 426/2; 426/60; 426/531; 426/661; 435/256.1

(57) ABSTRACT

A method of treating food waste by fermenting a raw material comprising the food waste by means of a koji mold to obtain a liquid substance. The koji mold is a koji mold belonging to genus Aspergillus capable of producing citric acid. The liquid substance comprises a citric add produced by the koji mold and has a pH of 5.5 or less. The method enables food waste to be used for feed without the problems usually associated with pig farming techniques using garbage. Additionally, the method allows food wastes to be preserved for a long time without putrefaction. Also offered is a feed composition comprising a treated food waste.

### BACKGROUND OF THE INVENTION

(1) Field of the Invention

The present invention relates to a method of treating food waste using an acidogenic koji mold.

(2) Description of the Related Art

The raising of pigs using garbage was common in the 1960's. However, improvements in living standards have raised the bar for pork quality, and such methods have largely fallen out of use today. The following three reasons can be given.

First, the oils contained in garbage can cause the fat in the pork to turn translucent or take on a yellowish tinge. This is the main reason.

Second, the development of blended feeds capable of quickly fattening pigs has made the raising of pigs using garbage unattractive due to the fact that garbage has an unstable nutritional content and therefore requires a longer fattening time. Third, garbage normally has a high water content of about 80%, so that it easily putrefies and is difficult to store.

On the other hand, the prospect of future feed shortages has led to establishment of the Food Recycling Law, and to renewed interest in the reuse of garbage. The following two methods have been tried in order to solve the above problems.

Fermentation by Lactic Acid

This method involves adding lactic acid bacteria to garbage to cause anaerobic fermentation, thereby holding the pH at 4 or less and preventing putrefaction, and allowing it to be fed directly to pigs. The advantage is the low processing cost. Conventional liquid feeding techniques almost all rely on lactic acid fermentation. However, this method has the following problems.

1. In the anaerobic state, the feed can be inadequately stirred, in which case some of it may undergo methane fermentation, which is not appropriate for feeds.
2. Lactic acid bacteria cannot easily decompose oils, which tends to reduce the meat quality.
3. The feed content is often inconsistent, making it necessary to supplement nutrients using existing feeds, which makes the work bothersome.

Heat Drying

This method involves heating garbage to dry to a water content of 15% or less, enabling it to be used as a highly preservable feed.

However, this method has the following problems.

1. Since the process requires garbage, which has a high water content, to be dried by heating, it involves very high processing costs often exceeding 100 US dollars per ton of garbage processed.
2. While being heated, the oils contained in the garbage can oxidize, thus reducing their quality.
3. Since the resulting feed has a high oil content (normally about 20%), no more than 10% can be added to blended feeds.

Due to the above-described problems, no highly economical garbage feeding methods have been developed until now.

Methods of fermenting food waste using koji mold have also been proposed (see WO 00/67588, JP 2002-142688A and JP 2002-336822A). In conventionally proposed methods, the putrefaction of organic substances in the fermentation process has been prevented by drying. That is, in conventional methods, the water content of the raw material is lowered (e.g., to a water content of 60% or less) and the water content is also held low during the fermentation process. Therefore, when using a raw material of high water content such as garbage, the water content must be reduced such as by adding another raw material that is dry before subjecting it to fermentation. Furthermore, the final product obtained by conventional methods consists of a solid or granules with a very low water content, such as 20% or less, in order to ensure preservability. Therefore, its transport involves difficulties in that manual labor is required for handling.

### BRIEF SUMMARY OF THE INVENTION

Therefore, the present invention offers a method of using food waste for feeds which solves the problems of conventional garbage-based pig farming. Additionally, the present invention offers a method of preserving food waste without allowing it to putrefy.

Furthermore, the present invention offers a feed containing treated food waste.

Since the treated food waste of the present invention contains citric acid, it is a suitable format for preserving food waste of high water and organic content without putrefaction. Additionally, since citric add is produced by koji mold in the fermentation process, the citric add is integrated into the food waste and is infused into the entire volume thereof.

Additionally, since garbage is normally not liquid, for examples, having a viscosity of 10,000 mPa·s or more, it must be handled manually during for transport. With the present invention, the garbage can be made liquid. Therefore, the treated garbage obtained in the present invention can be pumped and is therefore easily transported.

Furthermore, because it is liquid, contaminants contained in the garbage can be easily separated.

Accordingly, the present invention offers a method of treating food waste, comprising fermenting a raw material comprising food waste by means of koji mold to obtain a liquid substance. The koji mold is a koji mold of genus Aspergillus capable of producing citric add. Use of a koji mold having a high citric acid production capability makes the fermentation process safe due to the preservative effect of the citric add. Examples of koji molds having the ability to produce citric acid used in the present invention include Aspergillus awamori, Aspergillus inuii, Aspergillus ryukyuensis and Aspergillus saitoi. In the present invention, Asp. awamori kawachii (Kawachi Genichiro Shoten) is preferably used. While this koji mold secretes large amounts of extracellular enzymes, they are most effective in an acidic environment, so the koji mold has the advantage of being extremely resistant to acidity.

Additionally, the present invention relates to use of a koji mold of genus Aspergillus capable of producing citric acid in a method of treating food waste by fermenting the food waste by means of the koji mold of genus Aspergillus to obtain a liquid substance containing citric add produced by the koji mold and having a pH of 5.5 or less.

Furthermore, the present invention relates to a food waste treating agent comprising a koji mold of genus Aspergillus capable of producing citric acid for fermenting the food waste by means of the koji mold of genus Aspergillus to obtain a liquid substance containing citric add produced by the koji mold and having a pH of 5.5 or less.

In another embodiment, the present invention offers a method of treating food waste, comprising a step of mixing the food waste and a koji mold of genus Aspergillus having the ability to produce citric acid, a step of adjusting the water content of the mixture, and a step of aerobically fermenting the mixture to produce a liquid substance containing citric acid produced by said koji mold and having a pH of 5.5 or less. In a further embodiment, the present invention offers a method of treating food waste, comprising a step of mixing a raw material comprising food waste and a koji mold of genus Aspergillus having the ability to produce citric acid, a step of adjusting the water content of the mixture, and a step of aerobically fermenting the mixture to produce a liquid substance containing citric acid produced by said koji mold and having a pH of 5.5 or less, wherein only koji mold, especially koji mold of genus Aspergillus, is used as the microbe for the fermentation. Preferably, the water content of the mixture is adjusted to at least 75%, more preferably to at least 80%.

While putrefactive bacteria cannot easily grow in an environment of pH 5.5 or less and most microbes including E. coli cannot tolerate conditions of pH 4 or less, the koji mold used in the present invention is capable of growing in relatively strong acidic conditions. Therefore, the liquid substance obtained in the present invention preferably has a pH of 5.5 or less, more preferably pH 4.5 or less, and most preferably pH4 or less.

The water content of the raw material containing the food waste is preferably at least 70%, more preferably at least 75%, and most preferably at least 80%. Thus, while food waste usually contains large amounts of water, it can be used directly as is. Additionally, food waste normally has a high viscosity, for example, a viscosity of at least 10,000 mPa·s. However, the method of the present invention is capable of lowering the viscosity of the raw material to make the viscosity of the treated product 5,000 mPa·s or less, and in particular, 4,000 mPa·s or less.

The food waste used in the present invention is preferably raw garbage.

Fermentation is preferably performed aerobically by aeration. The fermentation conditions are set so as to produce enough citric add to obtain the desired pH. The conditions should preferably be such that koji mold, preferably koji mold having the ability to produce citric acid, grows dominantly. For example, the culture should have a temperature of at least 4° C. and the rate of aeration should preferably be at least 0.08 liters/minute, preferably at least 0.8 liters/minute for every kilogram of raw material.

The treated food waste offered by the present invention is preferably added to a starchy feed composition and fed to livestock, preferably as a feed for raising pigs. The invention allows for liquid feeding techniques, and the farming method of the present invention includes a method of producing pigs for pork.

Furthermore, the present invention offers a liquid substance for use in a feed, containing citric acid produced by a koji mold of genus Aspergillus and a food waste fermented by koji mold, having a viscosity of 2,000-5,000 mPa·s, preferably 3,000-4,000 mPa·s, and a pH of 5.5 or less. Preferably, the substance fermented by koji mold contains koji mold grown dominantly.

Preferred examples of the steps in the method of the present invention are described below.

1. Garbage such as kitchen scraps, leftovers, tofu lees, tea dregs, food past their expiration date and water used to rinse rice should be used as the raw material.
2. At least 108 acidogenic koji mold spores are added per kilogram of garbage.
3. If the water content is too low, water is added to adjust the water content to at least 80%.
4. The garbage to which the koji mold was added is aerated to grow the koji mold. The garbage is liquefied by the enzymes produced by the koji mold. Additionally, the citric acid produced by the koji mold makes the pH of the garbage 4 or less. Putrefaction is prevented by this reduction in pH. Furthermore, the oils contained in the garbage are decomposed by the koji mold.
5. A feed containing large amounts of starchy matter is added to the fermented garbage in which the koji mold has grown, so as to adjust the water content to 60%-90%, more preferably about 75%.
6. This feed is directly fed to pigs in the liquid state.

The present fermented feed accelerates the immune resistance of pigs and reduces their rate of mortality by disease.

The method of the present invention is capable of overcoming all of the problems of conventional garbage-fed pigs. In other words, it has the following effects.

1. It prevents putrefaction of garbage having a high water content at a low cost.
2. It prevents reductions in meat quality caused by the oils in garbage.
3. It prevents reductions in growth caused by inconsistencies in nutritional content.

The present invention also has the following effects.

4. It achieves high meat quality greatly exceeding that of pork raised by feeding conventional blended feeds.
5. It increases immune resistance and thereby prevents reduced yields due to deaths caused by disease.

### BRIEF DESCRIPTION OF THE DRAWINGS

FIG. 1 is a graph showing the average body weight change in Example 1.

FIG. 2 is a graph showing the change in feed component in Example 1.

FIG. 3 is a pie chart showing the results of a test of odor prior to eating in Example 3.

FIG. 4 is a pie chart showing the results of a test of meat color in Example 3.

FIG. 5 is a pie chart showing the results of a test of fat color in Example 3.

FIG. 6 is a pie chart showing the results of at test of tenderness in Example 3.

FIG. 7 is a pie chart showing the results of a taste test in Example 3.

FIG. 8 is a pie chart showing the results of a test of odor after eating in Example 3.

FIG. 9 is a pie chart showing the results of a test of overall preference in Example 3.

### DETAILED DESCRIPTION OF THE INVENTION

Preferred embodiments of the present invention shall be described below. Garbage consisting, for example, of kitchen scraps and leftovers, is seeded with spores of acidogenic koji mold to a concentration of 10⁸ per gram. Here, the garbage can be crushed in order to allow the mixture to be more complete. After seeding with koji mold, aeration is performed to promote the growth of the koji mold.

After 24 hours, the enzymes secreted by the acidogenic koji mold almost completely liquefies the garbage. With further growth, the acidogenic koji mold secretes citric acid, and aeration causes the garbage to be stirred, as a result of which the pH of the garbage is made uniformly 4 or less overall. Additionally, during this koji mold growth stage, the oils contained in the garbage are broken down.

The above steps are repeated as necessary until a weight appropriate for transport is reached. For example, it can be sent out upon reaching a volume suitable for transport by a tank lorry.

The resulting product can be freed of contaminants using conventional contaminant separation equipment. In this case, since the treated garbage is in liquid form, contaminant separation can be performed much more effectively than if contaminant separation were performed before the treatment.

A starchy feed material (such as maize) is added to the fermented garbage which has reached the appropriate weight, to adjust the water content preferably to 70-80%, and more preferably to 75%.

The resulting feed composition is fed to adolescent pigs of body weight in excess of 30 kg, and pigs are fattened to a body weight at time of shipment of about 100 kg basically using only this feed composition. On the other hand, during this time, conventional feeds can be added as nutritional supplements, and the feed can be changed to a conventional blended feed beginning one month prior to shipment.

As a result, the growth promotion function of koji mold enables the pigs to be shipped in 6 to 8 months after birth, which is no different from the standard for nutritionally enhanced feeds, without the need for adjusting the nutrient content.

Additionally, the pork produced in this way is not susceptible to the yellowing or softening of the fat which is common in conventional garbage-fed pigs.

Furthermore, the action of the koji mold reinforces the cell membranes, resulting in a flavorful pork with little drip.

### EXAMPLE 1

3.5 kg of seed koji of acidogenic koji mold (Asp. awamori) were added to 350 kg of kitchen scraps and leftovers discarded by 20 restaurants, adjusting the number of spores to 5×10⁹ per gram, and aeration was begun. The water content of the raw material was 80%.

After 24 hours, the garbage had mostly liquefied, and the pH had decreased to 3.9. The viscosity fell from 11,000 mPa·s prior to treatment to 3200 mPa·s after treatment (B-type viscometer by Tokyo Keiki).

Aerated cultivation was continued by adding 350 kg of garbage and 350 g of seed koji to this fermented liquid daily over a period of 4 days, during which time the pH was always 4 or less and no putrefaction was observed.

115 kg of maize was added to 1400 kg of this fermented liquid to adjust the water content to 75%, and the resulting feed was fed to 15 four-month old Berkshire pigs.

Additionally, as a control group, 15 four-month old Berkshire pigs were fed conventional blended feeds.

Thereafter, similar operations were continued until their body weight exceeded 100 kg, after which they were slaughtered and inspected for their meat quality.

The change in body weight is shown in FIG. 1. The test portion shows the change in average body weight of pigs fed the feed composition of the present invention. The control portion shows the change in average body weight of pigs fed a normal blended feed composition.

During this time, the pigs were fed without restriction, as a result of which they consumed 20% of their body weight in feed.

Additionally, as is clear from the graph, the pigs grew no differently from the pigs fed conventional blended feed, despite not being fed any feeds other than fermented garbage and maize.

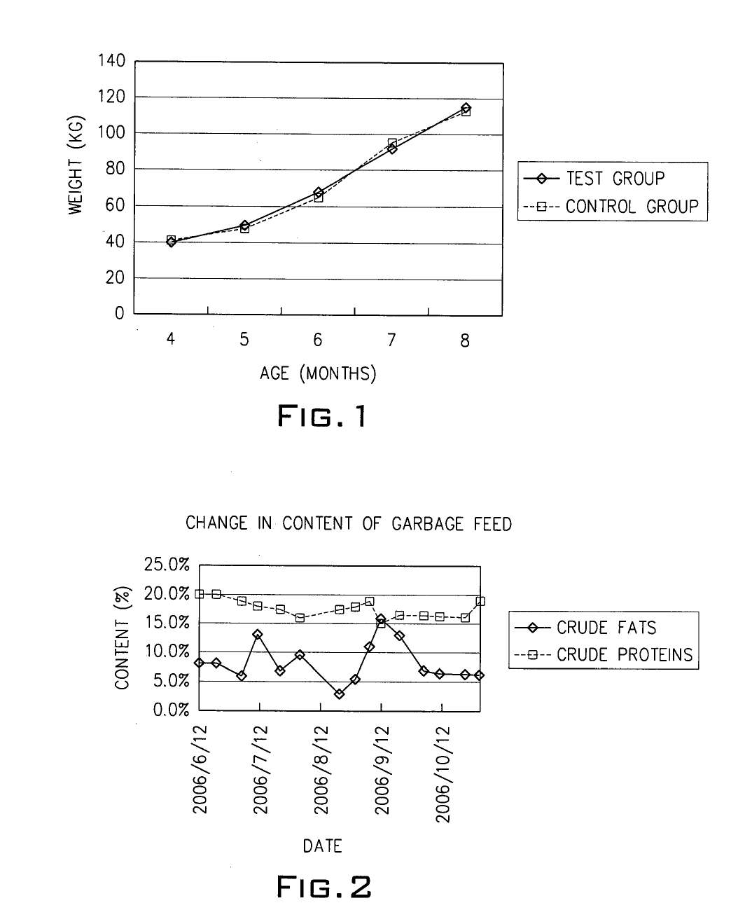

The change in feed content during this time is shown in FIG. 2. The component % is the proportion in dry weight.

Thus, while there were large deviations in the composition of the feed composition of the present invention, the pigs in this test achieved the standard body weight for shipment of 110 kg in 8 months, without much difference from pigs fed conventional blended feeds, as is clear from FIG. 1. This can dearly be attributed to the effects of the koji mold treatment, namely, the growth promoting effect, of the feed composition according to the present invention.

Furthermore, during this time, none of the pigs were lost to disease.

### EXAMPLE 2

The immune resistance improvement effect of the present feed composition was tested in mice.

Twelve mice were divided into two groups of six. The test group was given the fermented garbage feed composition of Example 1 without restriction, and the control group was given the mixed feed composition indicated below.

| | |
|---|---|
| Casein | 11.0% |
| Barley bran | 15.6% |
| Wheat bran | 6.0% |
| Wheat gluten | 8.4% |
| Leucine | 0.2% |
| Lysine | 0.2% |
| Phenylalanine | 0.3% |
| Corn starch | 26.2% |
| Corn oil | 15.0% |
| Cellulose powder | 1.5% |
| Sucrose | 10.0% |
| Mineral mixture | 3.5% |
| Vitamin mixture | 2.0% |

In the test, the twelve mice in the test group and the control group were fed the respective feed compositions for 3 days, given subcutaneous injections of the Sarcoma180 virus, then further raised for a period of 14 days, and subjected to analysis.

The results are shown below.

TABLE 1

| | Cancer Weight | Liver Weight | Spleen Weight |
|---|---|---|---|
| Control | 0.280 ± 0.086 | 1.344 ± 0.079 | 0.160 ± 0.053 |
| Test | 0.166 ± 0.093 | 1.219 ± 0.159 | 0.120 ± 0.023 |

As is clear from this table, the weight of cancer is clearly reduced in the test group as compared with the control group, while the weight of the liver does not increase, thus confirming that the fermented garbage feed composition of the present invention is not toxic and promotes immune resistance.

### EXAMPLE 3

Next, the results of taste tests of pork from the pigs in the test of Example 1 shall be shown below.

Participants in Taste Test:
20 male subjects
24 female subjects

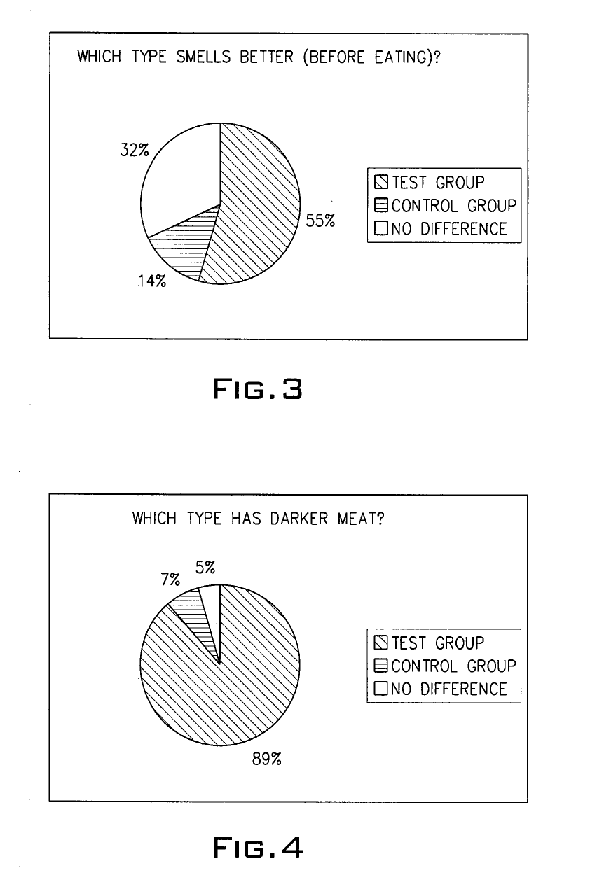

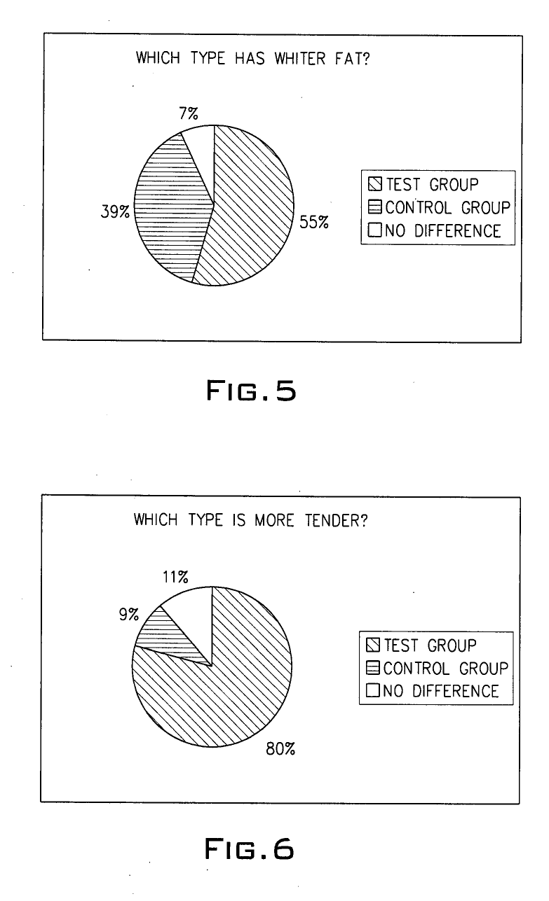

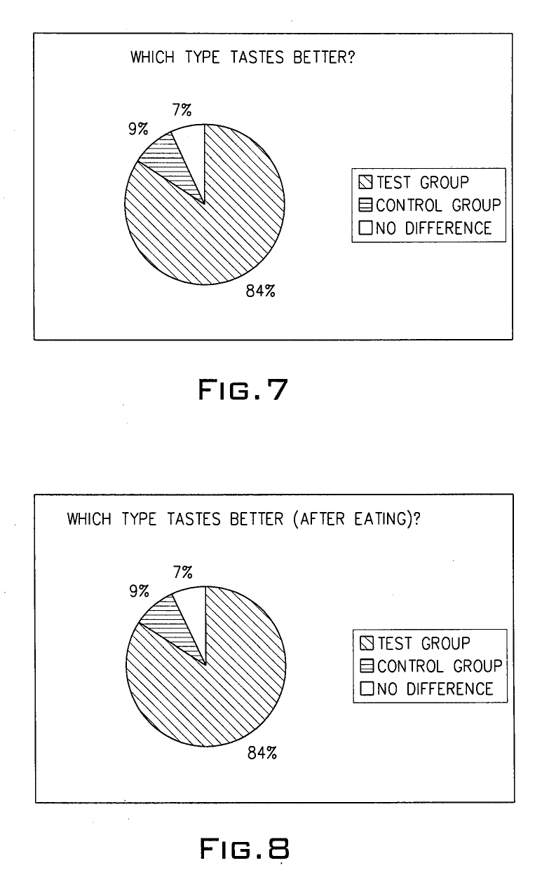

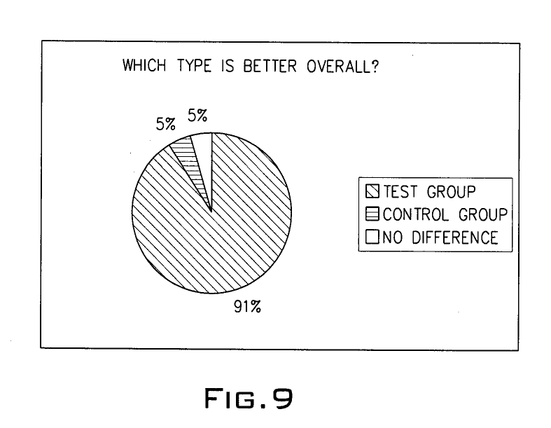

The results of judgments of the odor prior to eating, meat color, fat color, tenderness, taste, odor after eating and overall preference are shown respectively in FIGS. 3-9.

As is clear from the pie charts of FIGS. 3-9, the pork from pigs fed the fermented garbage feed composition received much higher evaluations than pork fed on conventional blended feeds.

While the major issue for previous pig farming techniques using garbage lay in how to achieve anything close to the quality of the pork from pigs fed blended feeds, the present feed production method is revolutionary in that it actually reverses the scales by producing pork of a quality greatly exceeding that of conventionally raised pigs.

### EXAMPLE 4

Further, the difference in drip in the pork from pigs fed the feed of Example 1 was considered.

### Testing Method

Test Group: Ten 60-g slices of pork loin produced by the present technique
Control Group Ten 60-g slices of pork loin produced with conventional blended feeds

The above-described pieces of pork were placed in a hot water tank with the temperature held at 91° C. to 93° C., and the change in weight after 15 seconds was measured.

TABLE 2

| | Before Treatment | After Treatment |
|---|---|---|
| Test | 60.7 g | 18.4 g |
| Control | 60.1 g | 42.9 g |

As is clear from this table, the pork produced with the present technique can be seen to have 10% less drip than pork produced using conventional blended feeds.

1. A method of treating food waste, comprising:
fermenting a raw material comprising food waste by means of koji mold to obtain a liquid substance;
wherein said koji mold is a koji mold of genus Aspergillus capable of producing citric acid; and
said liquid substance contains citric acid produced by said koji mold and has a pH of 5.5 or less.

2. A method in accordance with claim 1, wherein said raw material comprising food waste has a water content of at least 80%.

3. A method in accordance with claim 1, wherein said liquid substance has a viscosity of 5,000 mPa·s or less.

4. A method in accordance with claim 1, wherein said raw material has a viscosity of 10,000 mPa·s or more.

5. A method in accordance with claim 1, wherein said fermentation is aerobic fermentation.

6. A method in accordance with claim 1, wherein said fermentation takes place under aeration.

7. A method in accordance with claim 1, for the purpose of preserving food waste.

8. A method in accordance with claim 1, for the purpose of producing a feed composition.

9. A treated food waste obtained by a treatment method in accordance with any one of claims 1-8.

10. A feed composition comprising a treated food waste in accordance with claim 9.

11. A feed composition in accordance with claim 10, further comprising a starchy feed composition.

12. A feed composition in accordance with claim 10, for raising pigs.

13. A method of farming livestock comprising feeding the livestock a feed composition in accordance with claim 10.

14. A liquid substance comprising citric add produced by koji mold of genus Aspergillus and a food waste fermented by the koji mold, having a viscosity of 2,000-5,000 mPa·s and a pH of 5.5 or less.

15. A liquid substance in accordance with claim 14, for use as a feed composition.

16. Use of a koji mold of genus Aspergillus capable of producing citric acid in a method of treating food waste by fermenting the food waste by means of the koji mold of genus Aspergillus to obtain a liquid substance containing citric acid produced by said koji mold and having a pH of 5.5 or less.

17. A food waste treating agent comprising a koji mold of genus Aspergillus capable of producing citric add for fermenting the food waste by means of the koji mold of genus Aspergillus to obtain a liquid substance containing citric acid produced by said koji mold and having a pH of 5.5 or less.
